(19) United States
(12) Patent Application Publication
(10) Pub. No.: US 2025/0231788 A1
(43) Pub. Date: Jul. 17, 2025

(54) SYSTEM AND METHOD FOR A REMOTE THERAPY PLATFORM FOR COLLABORATIVE ACTIVITIES

(71) Applicant: MainSquare Co., San Francisco, CA (US)

(72) Inventors: Julia Neizonek, San Francisco, CA (US); Aaron Blacker, Stanford, CA (US); Murilo Cruz, Indaiatuba (BR); Thomas Fanella, New York, NY (US)

(21) Appl. No.: 19/021,942

(22) Filed: Jan. 15, 2025

Related U.S. Application Data
(60) Provisional application No. 63/621,053, filed on Jan. 15, 2024.

(51) Int. Cl. G06F 9/455; G16H 40/67

(52) U.S. Cl. CPC G06F 9/45558; G16H 40/67; G06F 2009/45595

(57) ABSTRACT

A system and method for collaboration by multiple participants during a digital activity session that includes a remote server; at least one virtualized computing environment instantiated on the remote server; a remote shared application instance, like a browser instance, executed within the at least one virtualized computing environment; and a set of participant clients connected to the remote shared application instance through an input interaction channel that communicates inputs from the set of participant clients to the remote shared application instance and an application media stream which communicates media from the remote shared application instance to the set of participant clients.

### CROSS-REFERENCE TO RELATED APPLICATIONS

This application claims the benefit of U.S. Provisional Application No. 63/621,053, filed on 15 Jan. 2024, which is incorporated in its entirety by this reference.

### TECHNICAL FIELD

This invention relates generally to the field of digital tele-health applications, and more specifically to a new and useful system and method for a remote therapy platform for collaborative activities.

### BACKGROUND OF THE INVENTION

In recent times, remote teleconferencing has seen a significant rise in popularity, transforming the way healthcare and education services are delivered. However, there are many challenges and issues when using remote tele-health tools, particularly in the realm of tele-health and remote education where activity-based interventions and resources are deployed.

One prominent challenge stems from the fact that most digital tools within the communication and collaboration space are primarily designed for corporate or business workers and workflows and may not cater to individuals with limited technical capabilities or who rely primarily on non-verbal modalities for communicating or displaying psychological presentations. For example, in the case of children, standard communication and collaboration tools have challenges achieving engagement. Additionally, children may lack the necessary technical knowledge to efficiently navigate and manage different applications on a computer, as well as perform basic actions with digital tools. Consequently, their ability to effectively participate and engage in tele-health or remote education experiences can be hindered. As another example, for the case of adults, higher acuity medical protocols for treating mental health disorder may call for direct deployment of measurement-based screeners, manualized worksheets, and the like, during a healthcare encounter—which isn't easily possible with modern healthcare teleconferencing technology, which utilizes the same aforementioned technology as business environments. This technology neither embeds the relevant protocols for easy retrieval, enables saved progress and workflows for specific patients for clinical approaches, nor enables interactive, engaging deployment of relevant tools towards achieving treatment goals.

Moreover, the utilization of different digital tools for therapy and interventional services further complicates matters. Screen sharing is a commonly employed method where the therapist, through sharing their screen, can attempt to make use of a digital application on their device. In many cases, the therapist must have the patient provide instructions on how they would interact with the activity. However, this approach presents several limitations. For example, the patient is restricted in their ability to directly interact with activities and interventions or provide important non-verbal clinical feedback due to the one-sided nature of screen sharing. Alternative methods, like providing the child with mouse control to the adult's computer presents privacy and HIPAA concerns given clinical notes are often open somewhere on the clinician's computer. Collaborative whiteboards are also common but lack modularity and functionality to support many intervention approaches, modalities, and practices, which may prescribe incorporation of specific content, like psychoeducational materials, homework interventions (e.g., thought records, dairy cards, etc.), and worksheets into the patient encounter.

Thus, there is a need in the tele-health applications field to create a new and useful system and method for a remote therapy platform for collaborative activities. This invention provides such a new and useful system and method.

### BRIEF DESCRIPTION OF DRAWINGS

FIG. 1 is a schematic representation of using an exemplary platform of the system and method.

FIG. 2 is a schematic representation of a system variation.

FIG. 3 is a schematic representation of a system for an activity session with N participant clients using peer-to-peer communication.

FIG. 4 is a schematic representation of a system for an activity session with N participant clients using an intermediary media server.

FIG. 5 is a schematic representation of the system as a scalable computing platform.

FIG. 6 is a schematic representation of using an exemplary platform of the system and method using artificial intelligence (AI).

FIG. 7 is a schematic representation of using an AI service.

FIG. 8 is a schematic representation of using an AI service with trained AI models.

FIG. 9 is a schematic representation of two states of a group activity session where a participant C is moved to a breakout session to work on a digital activity individually until a participant may join them.

FIGS. 10A and 10B are exemplary screenshots of an activity session application.

FIG. 11 is an exemplary screenshot of an activity session application using a video conferencing user interface design.

FIG. 12 is a representation of operation of control management for multiple inputs.

FIG. 13 is a flowchart representation of a method variation.

FIG. 14 is a schematic representation of management of sessions.

FIG. 15 is a schematic representation of management of activities during a session.

FIG. 16 is an exemplary system architecture that may be used in implementing the system and/or method.

### DETAILED DESCRIPTION OF THE EMBODIMENTS

The following description of the embodiments of the invention is not intended to limit the invention to these embodiments but rather to enable a person skilled in the art to make and use this invention.

### 1. Overview

The systems and methods herein describe a digital activity platform for remote collaboration by multiple participants during a digital activity session. The systems and methods may also integrate remote communication like video and/or audio communication within the collaborative digital activity session.

The systems and methods can have particular applicability for use in therapy sessions. In some variations, the systems and methods may function to enable a virtual therapy room or space. The virtual therapy rooms of the systems and methods may especially be useful during the treatment and remote care sessions for patients where care providers may want to be able to have the patient engage with various digital tools and activities in an interactive, personalized, and collaborative environment. For example, the systems and methods may be used to provide an interactive digital space for therapists to engage remotely with patients, in particular children, where a variety of different personalized activity resources can be easily used collaboratively during video and/or audio sessions.

The systems and methods may also have utility when used with non-child participants such as when there is one or more facilitator (e.g., therapist, trainer, teacher, or other type of facilitator) interacting with a subject participant (e.g., a patient, trainee, student, or some other participant that generally is being assisted or overseen by the facilitator). The systems and methods may also have applications in a variety of areas. For example, the systems and methods may have more general applications in the telehealth space for pediatric primary care, imaging display for orthopedics, and/or other fields. The systems and methods can similarly have applications in other areas.

The systems and methods preferably involve hosting a remote shared browser instance that is streamed to the participants. The participants can use an activity session application (e.g., accessed/running on their own browser) for collaborating with different activities, where some of those activities may run on the remote shared browser instance. The hosted shared browser instance can be used to facilitate access to a variety of different digital activities during a session.

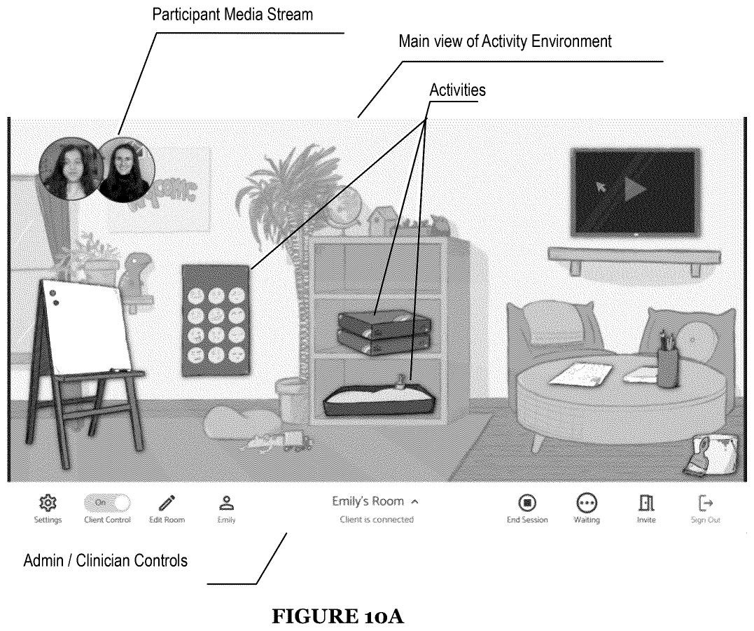

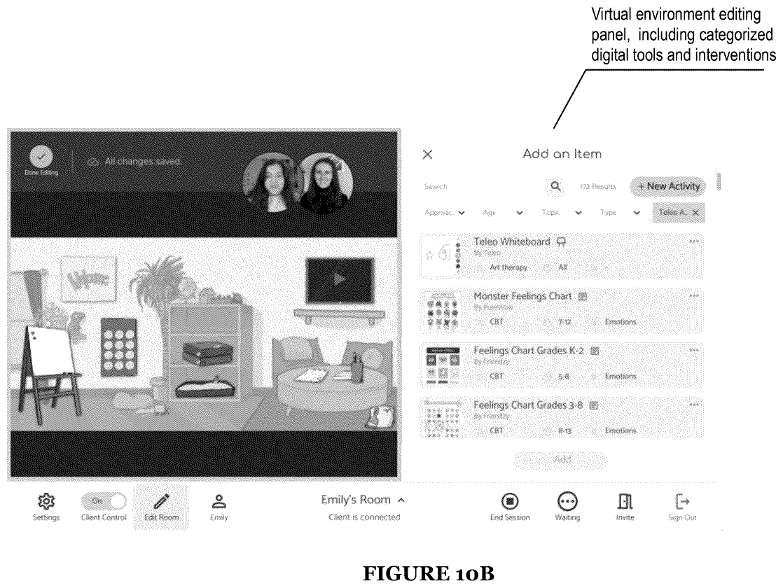

In one exemplary implementation, the systems and methods may present access to the different digital activities through a graphical representation modeled off of a physical space as shown in FIGS. 10A-10B, but other representations may alternatively be used.

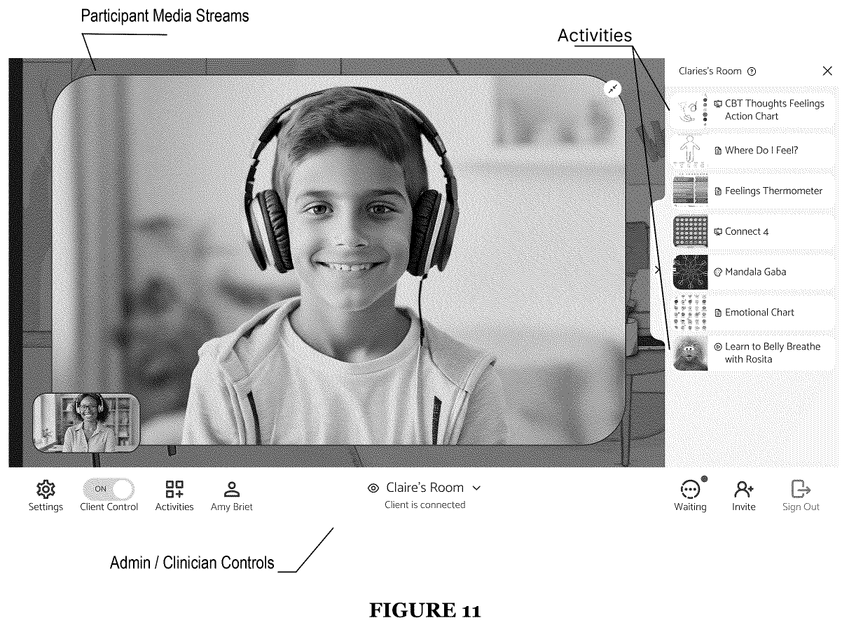

In another example, different digital activities may be presented and/or accessed alongside a video chat interface as shown in FIG. 11. The systems and methods can accommodate integration of web applications accessible over the internet, making the systems and methods immediately usable alongside the wide diversity of web applications available.

As one challenge, therapists and/or facilitators may use a variety of different digital tools when working with clients. Many of these digital tools used are often accessed over the internet. However, they are designed for use by a single participant accessing from a single computer. The systems and methods can enable shared collaborative use of such internet-accessible tools despite these digital tools not natively supporting such capabilities.

A control management service used within the remote shared browser instance facilitates streaming input control from the different participants and coordinating exchange of control between participants and enforcement of changing input permissions. The control management service may function to enable collaborative control of a web application that is intended for control by a single participant. The systems and methods may be operated in such a way that can protect data privacy. The systems and methods may additionally enable integration for AI-augmented monitoring, tracking, recommendations, and content generation.

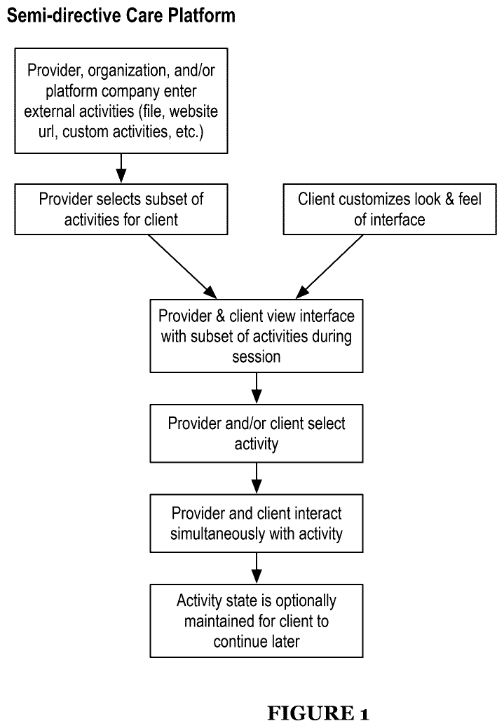

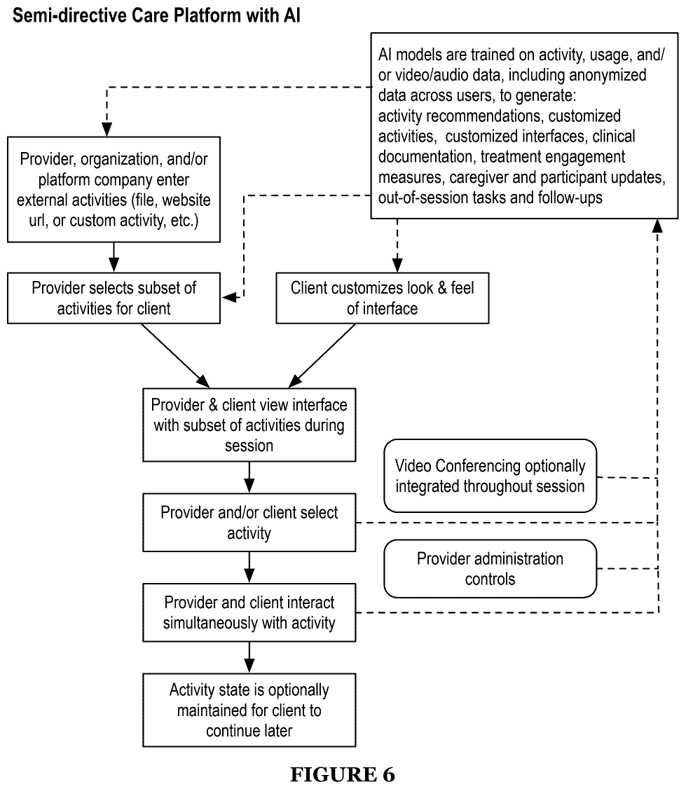

As shown in FIG. 1, the systems and methods may be used such that the provider can easily manage use of different activity sessions. The provider can select different activities (internal or external ones), start a session with a subject participant and engage with different activities. In some implementations, the representation of navigation to the different activities can be presented as an environment with activities represented by tangible graphics as shown in FIGS. 10A and 10B. The presentation of this environment and the activities may be customized by participants. As discussed in some variations an AI service may be used to augment use of the platform as shown in FIG. 6.

Furthermore, the activity state of a particular activity may be optionally maintained such that a participant may revisit the activity in such a maintained state at a later point. For example, activity state information for a given activity session application or an activity session in a remote shared browser instance may be saved. This may include saving image snapshots, saving a state object (e.g., JSON object), or saving state in any suitable manner. In one variation, a remote shared browser instance state may be preserved between participant activity sessions. In this way, an activity running on the remote shared browser instance can be revisited by a participant even after the end of their session.

Use of the systems and methods will generally involve multiple participants. The participants will generally have different roles. However, the system and method may be used in situations where there are no distinct roles. Herein, the systems and methods are primarily described as: used with two or more participants, with one or more participants being a provider or “facilitator” participant (i.e., a provider or facilitator of some service) and additional participants being subject participants (i.e., subjects). A provider participant could be a therapist, healthcare specialist, teacher, trainer, or some other person facilitating a session. A subject could be a patient, student, trainee, observer, or a beneficiary of the services provided by the provider. In some applications, the subjects could be children or adolescents, though use of the systems and methods is not limited by age.

As discussed, the systems and methods may, in some exemplary applications, be used for child-focused therapy. The systems and methods could be used to provide remote care sessions for mental health therapy, speech therapy, psychiatry, occupational therapy, medical patient education, remote educational interventions, employee training, and/or other treatments or sessions that can be provided through device-based interactions. In some alternative applications, the systems and methods may be used for other remote tele-health applications, for remote educational coaching/instruction, and/or for any suitable applications. In some situations, the systems and methods may be used in connection with in-person care, where the systems and methods enable use of the digital activities in situations where, for example, there may be limited physical resources or strict cleaning requirements. The systems and methods accordingly may even be used on a single device or multiple devices with video streaming disabled but still leveraging collaborative capabilities, including collaborative capabilities using a remote browser instance or provided in other file-based/native applications. Herein, the systems and methods will primarily be described as and use examples based on the child-focused therapy use case. However, the systems and methods are not limited to just this use case, and, as one skilled in the art would appreciate, the systems and methods may be similarly applied in other use cases.

The systems and methods may provide a number of potential benefits. The system and method are not limited to always providing such benefits and are presented only as exemplary representations for how the system and method may be put to use. The list of benefits is not intended to be exhaustive, and other benefits may additionally or alternatively exist.

As one potential benefit, the systems and methods may provide a collaborative interface that can be used for child-focused care, where care providers often rely on multiple digital tools when working with a child subject. Furthermore, the number, type, and versions of digital tools that may be used may be ever changing, and the system and method provide a solution that can work with most browser-based activities and other activities, without depending on any adjustments to the digital activities themselves. The collaborative interface allows care providers to seamlessly access and utilize various digital tools, enhancing their ability to provide comprehensive and personalized care and support to children.

As another potential benefit, the systems and methods may enable shared remote control of different applications. For example, the systems and methods may allow multiple participants to share control of a web application originally designed for standard web browsers with a single source of input. This means that mouse and keyboard inputs can seamlessly switch between a provider and a subject participant, facilitating collaborative interactions during therapy sessions or other remote activities.

As another potential benefit, the systems and methods may provide personalization and flexibility, allowing care providers to utilize a wide range of tools and applications, particularly in child-focused therapy and care. This enables care providers to access and integrate various tools from different websites, enhancing the effectiveness and customization of the therapy and care provided. For example, in the system and method, a care provider may seamlessly utilize interactive games and educational materials of their choosing to cater to the specific needs (e.g., presenting clinical challenges) and interests of a specific child as well as the clinical approaches and protocols chosen by the facilitator. These activities may be used within an integrated digital platform that is child-friendly to use, adds provider-subject collaborative input control, and that provides a care provider administrative control over the session.

As another potential benefit, the systems and methods may function to protect personally identifiable information (PII) and/or protected health information (PHI). This can be achieved by utilizing virtualized computing environments that fully isolate participants from other sessions. Additionally, each virtualized computing environment can be deleted when a session is ended to ensure that related data is cleared. Running the browser on a remote server further reduces the risk of unintentionally leaking client PII or PHI such as internet protocol (IP) addresses and/or tracking cookies to third-party websites. For example, in the system and method, when a participant initiates a browsing session, a separate virtualized computing environment can be created to encapsulate all of the participant's activities within that session. This virtualized computing environment keeps the participant's PII and PHI completely isolated from any other sessions or processes running on the system. At the end of the session, the virtualized computing environment is automatically deleted, ensuring that any data or information associated with the participant is permanently removed. Such protections may be useful to make the digital platform compliant with relevant data privacy regulations (e.g., HIPAA compliance).

As another potential benefit, the systems and methods may enable sessions with opaque management of the activities. This feature is particularly important in therapy sessions with children, as it helps to enhance engagement and enable non-directive care, which is central to certain therapy approaches. Opaque management of activities allows for a more interactive and immersive experience, where therapists can guide a child through various digital tools and activities in a user interface optimized for clarity, engagement, and ease of use, without exerting direct control. For example, therapists can select and present different personalized activity resources while the child actively chooses among them and engages with them in a collaborative manner during video and/or audio sessions. This approach promotes client engagement and success, as it empowers children to take an active role in their therapy sessions (e.g., child-led, client-led, or non-directive therapy sessions) while still focusing the sessions on clinically beneficial interventions. The systems and methods enable this over remote digital interactions.

As another potential benefit, the system and method may enable provider-managed permissions, allowing a provider to have control over the applications used within the virtual therapy room. With this capability, the provider can restrict input from the subject participant and/or control which application(s) are available or used during the session. This aspect enhances the provider's ability to tailor the therapy experience and ensure that the session remains focused and effective. For example, a therapist can limit the access of certain applications to prevent distractions or customize the activities based on the subject's needs. This feature provides flexibility and adaptability to optimize the therapy session for the best outcomes.

### 2. System

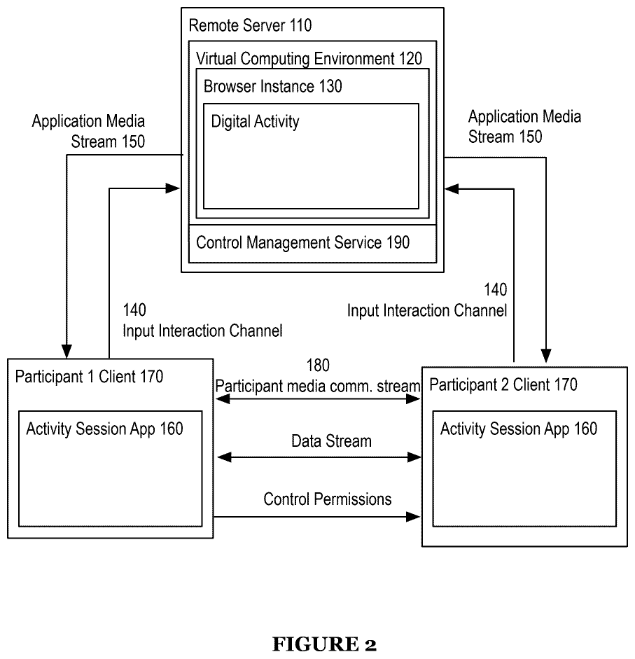

As shown in FIG. 2, a system for a remote therapy platform for collaborative activities may include a remote shared browser instance 130 operable on a remote server 110 and which includes an activity session application 160, a virtualized computing environment 120 where the browser instance 130 runs one or more activities of the activity session application 160; and a set of participant clients 170.

The system may additionally include an application media stream 150 and an input interaction channel 140 between the participant clients 170 and the shared browser instance 130. Some variations may additionally include integration with participant media communication streams 180, which functions to combine real-time video and/or audio communication with use of the activity session application 160. Additionally, in some variations, the system may include a control management service 190 that functions to facilitate collaborative shared control of activities used within the activity session application. As yet another possible addition, the system may include an artificial intelligence (AI) service 200 integrated with the system so as to provide automated tracking, evaluations, recommendations, and/or other automated actions. Some variations of the system may also include data integrations with electronic health record (EHR) data system(s) and/or biometric data integration, which may be used to alter the system and/or used in connection with the AI service 200.

Accordingly, in some variations, a system for enabling remote collaboration by multiple participants during a digital activity session may include: a remote server 110; at least one virtualized computing environment 120 instantiated on the remote server 110; a remote shared browser instance 130 executed within the at least one virtualized computing environment 120; and a set of participant clients 170 that are connected to the remote shared browser instance 130 through an input interaction channel 140 which communicates inputs from the set of participant clients 170 to the remote shared browser instance 130 and an application media stream 150 which communicates media from the remote shared browser instance 130 to the set of participant clients 170.

The system will also generally include an activity session application 160 that is operated at each of the set of participant clients 170. This may be a web application served to and operated at the participant clients 170 or native application run on a computing device of the participant. The activity session application can be the local application through which a participant receives a media stream of and interacts with a digital activity, run on the remote shared browser instance 130, using the input interaction channel 140.

In particular, the activity session application 160 can include action inputs that are configured to trigger activation of digital activities, wherein activation of at least one digital activity launches a digital activity on the remote shared browser instance 130. The media of the digital activity running on the remote shared browser instance 130 is streamed and presented in the activity session application of each participant client through the application media stream. In one implementation, the digital activity may be a web application or website URL that is loaded on the remote shared browser instance 130.

As an alternative description of a variation of the system, a system for enabling remote collaboration among multiple participants during a digital activity session, may include: a remote server 110 configured to host virtualized computing environments 120; at least one virtualized computing environment 120 instantiated on the remote server 110, wherein the virtualized computing environment 120 includes: a remote shared browser instance 130 configured to execute web-based activities, and a control management service 190 configured to manage input commands from a set of participant client devices 170, providing coordinated input control over the remote shared browser instance 130; instances of an activity session application 160 operating on the set of participant client devices 170, wherein the activity session application 160 provides a user interface for interacting with media of the web-based activities streamed from the remote shared browser instance; and wherein the instances of the activity session application 160 and the remote shared browser instance 130 are connected through at least: an input interaction channel connecting the participant client devices to the remote shared browser instance, enabling transmission of input commands from the client devices to the remote shared browser instance, and an application media stream configured to deliver media content from the remote shared browser instance to the activity session application on each of the participant client devices.

Furthermore, the set of participant media communication streams 180 may be configured to transmit video and audio data between the instances of the activity session applications 160 of the participant client devices 170, facilitating real-time communication among participants during the digital activity session. The application media stream 150 may or may not be included as part of media communication of the participant media communication streams 180. For example, in some variations, the shared browser instance 130 may be shared similarly to video shared by the other participants, other than the remote host may not receive media streams from participants.

The web-based applications can be accessed simultaneously by multiple participant client devices, with the application's interface being remotely visible (both visuals and auditory) and controllable (e.g., through coordinated control as discussed herein).

The activity session application may be configured to display video and audio communication between participants and to present shared content streamed from the remote shared browser instance. The activity session application may also provide a user interface to enable selection and activation of different activities (e.g., different web-based applications).

The system can also include a control management service 190 configured to manage and coordinate input from activity session applications 160 belonging to different participant client devices in a session, allowing for dynamic allocation of control over the web-based applications within the shared browser instance to facilitate interactive collaboration. Such a control management service 190 can be integrated to regulate control of the web-based applications of the remote shared browser instance 130.

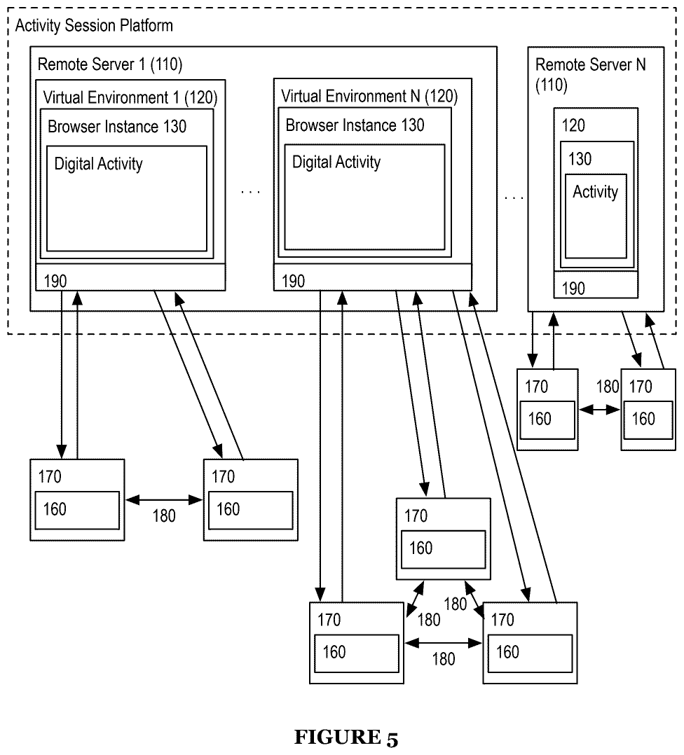

The system as described herein, primarily describes a system topology and features as implemented for one session. However, the system will generally be implemented as a scalable software service that enables many people to create accounts and run their digital service. The system can preferably be scalable to support different numbers of sessions and/or number of participants in each session. In particular, the number of remote servers 110 and/or the number of isolated virtualized computing environments 120 in each remote server 110 may be scaled. Accordingly, as shown in FIG. 5, a multi-tenant platform may include: one or more remote servers 110, each remote server 110 hosting one or more virtualized computing environments 120; wherein each virtualized computing environment 120 executes one or more remote shared browser instances 130; a set of participant client devices 170; each participant client device 170 may incorporate an activity session application 160; wherein each subset of participant clients 170 in a shared activity session are connected to a particular remote shared browser instance 130 of that shared activity session through an input interaction channel 140 that communicates inputs from the set of participant clients 170 to the remote shared browser instance 130 and an application media stream 150 that is a media stream that communicates media from the particular remote shared browser instance 130 to the set of participant clients 170. A given remote server 110 can scale the number of virtualized computing environments 120 to change the number of activity sessions supported by a single remote server. Similarly, a platform could scale the number of remote servers 110 to similarly scale support for changing the number of activity sessions.

The remote server 110 (“server”) functions as a computing resource used for running the virtualized computing environments 120 and browser instances 130. The server 110 may be dedicated or shared. Any sort of network accessible computing resource may alternatively be used. In some variations, the system may be implemented across a computing cloud using multiple servers 110 for managing a digital activity platform.

The number of servers no may be scaled to support multiple simultaneous activity sessions for different groups of participants. In general at least one server (or equivalent computing device/environment) is used in facilitating at least one activity session. In one scenario, the number of remote servers no can be increased or decreased to meet demand.

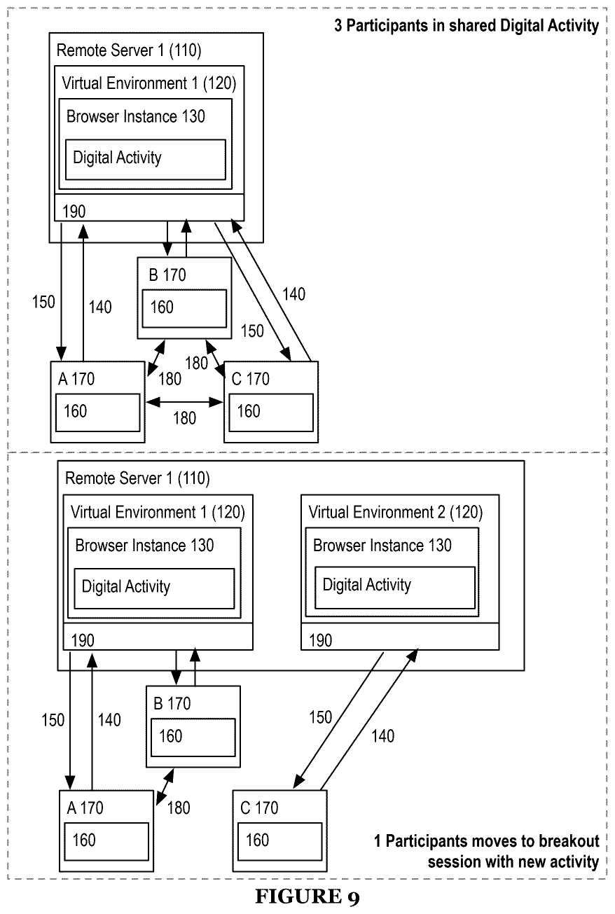

In another alternative scenario, two servers no may be used in facilitating a single activity session. For example, two different activities running in separate browser instance 130 may be used for an activity session to support features like “breakout sessions” where a facilitator may have different subject participants work individually while the facilitator moves between them as shown in FIG. 9.

The virtualized computing environment 120 functions as an isolated computing environment in which one or more different digital activities used within an activity session application may be run. More specifically, the virtualized computing environment may run a browser application instance in which one or more digital web activities are run. The virtualized computing environment 120 will generally run digital activities that are from 3rd party web applications or websites.

In one variation, an individual virtualized computing environment 120 may be used for each session, or at least each session with an activity that is shared and made collaborative. As such, an individual virtualized computing environment 120 may be initiated or selected for use for each session. In some variations, the virtualized computing environment may be used only for a single session. The environment 120 may be ended/deleted after a session. In some variations, however, the environment 120 may be maintained after a meeting of participants such that the state of the activity session can be re-established during a later meeting. As one such example, a virtualized computing environment 120 may be maintained for some time period after a session in case the participants come back. Alternatively, there could be a selectable option where a participant could request the session be maintained for some amount of time. For example, a therapist could save the state of activities for the next scheduled meeting.

As used herein, the term ‘virtualized computing environment’ characterizes any hosted computing environment that provides isolation between software applications and/or processes. Some examples may include virtualized computing environments implemented using containerization technologies such as Docker, or virtual machines running on a hypervisor. The term is intended to encompass any mechanism or technology capable of creating such isolated environments. Accordingly, in some variations, the isolated computing environment 120 is a container environment. A container environment can be an isolated container using some containerization technology such as Docker. In another variation, an isolated computing environment 120 may be a virtual machine environment running on a hypervisor.

The remote shared browser instance 130 (or more simply browser instance) functions as a browser application used to run one or more activity applications or websites. Use of a browser instance 130 can allow any website or web application to be run in a computing environment that can be shared and made collaborative. The activities may be web applications but could be any suitable website or web-accessible resource. Operating as web applications can make sharing and use of the digital activity platform easy for participants. For example, participants could simply join a session by visiting a shared link in a browser client, where the link may load an activity session application 160 or display the streamed content of the browser instance 130 directly.

A browser instance 130 will generally operate within the virtual computing environment 120 hosted on the server 110. Multiple distinct instances of the browser instance 130 may be run concurrently, either as individual applications within one virtualized computing environment 120 or as separate instances across multiple virtualized computing environments 120 (on the same or different servers 110). In situations where privacy or multi-tenancy is a lower priority, the system may be implemented where the browser instance 130 is run directly in the remote server 110.

The browser instance 130 may be customized or augmented. For example, the user interface and chrome of the browser instance 130 may be altered to simplify the functionality. In some implementations the navigation bar and tool access may be disabled. Additionally the browser instance 130 may be modified to include additional user interface elements specific to the digital activity platform. In some variations, the viewable portion of the browser (e.g., the view that is streamed and visible to participants) may be restricted to a subregion of the browser instance 130. For example, just the main view of the current activity may be streamed through the application media stream 150.

The browser instance 130 may include a main application view where a currently active website (or web application) is presented. The browser instance 130 can preferably maintain states of multiple application views. In some variations, the application views (possibly modeled in the browser instance 130 as distinct browser windows or tabs) are presented as modular application views thereby hiding the browser-based nature of the applications. In this way, the browser instance 130 may show a singular application view at a time. Other approaches may alternatively be used in other implementations.

The browser instance 130 may be a modified browser. However, in another implementation, the browser instance 130 may be a native application implementing web views or web rendering to provide a substantially similar interface. In some variations, the system may include a more generalized application instance in place of a browser instance 130. As one example, an application instance could be a game that could be made cooperative by streaming its media and resolving shared inputs from multiple participants. As another example, the application instance could be a file or file viewing application.

In one alternative variation, the system may be operated with a different architecture that can enable the system to use one browser instance of a participant as a host or “main” browser instance. An activity can run natively within the host browser instance. As in the other scenario happening on a remote server 110, the input interaction channel 140 and application media stream 150 may allow streaming of the activity to the other participant, and for receiving input interactions from the other participants. This variation can provide a more client-to-client communication topology and may not need a remote server 110 or virtualized computing environment 120.

The input interaction channel 140 functions as a data connection through which control input from one or more participants is transferred from a participant client device to the virtualized computing environment 120 to control an application loaded in the browser instance 130. There is preferably a data channel for each participant to supply input interactions. The input interactions may include any suitable type of computer input such as keyboard input and mouse input, but may additionally include controller input (e.g., for specialized computing peripherals), video input, audio input, file/data upload inputs, and/or other types of input. Input supplied by multiple participants can be managed and coordinated by the control management service 190.

The input interaction channel 140 may be implemented through a persistent data stream. Alternatively, the input interaction channel 140 may be implemented using an endpoint (e.g., an API endpoint) that can be a recipient of input updates from participant clients.

The input interaction channel 140 for each participant is preferably supplied as an input to the control management service, which functions to manage coordinating control supplied by multiple participants.

The input interaction channel may be a data stream through a persisted connection but could alternatively be data message communications transmitted in response to input by a participant. In some variations, the input interaction channel from each participant client is maintained continuously. In some variations, the input interaction channel may be communicated during active use of the browser instance and deactivated when the browser instance is not being used. However, in some situations, the input interaction channel may depend on who has active control of input in the browser instance 130.

In some variations, the server 110, the isolated container 120, and/or browser instance 130 may support multiple simultaneous input devices. For example, some operating systems support multiple simultaneous cursors or mice.

The application media stream 150 functions as a video and/or audio stream of the content of the browser instance 130 and application(s) loaded in the browser instance 130. The system can be configured to communicate and establish data connections for application media stream(s) to relay content from the remote shared browser instance 130 to an activity session application 160 on each participant client device 170. This can enable synchronized experience of the application loaded on the browser instance 130 as a collaborative environment. Streaming video of the browser instance 130 allows each participant to see, hear, or otherwise experience the current state of the application. Streaming media may additionally include streaming the cursor position.

In some variations, the application media stream 150 may be an independently managed media stream to the participant clients. This can be performed and set up separate from video/audio streams of the participants (e.g., the face-to-face media conferencing).

In another variation, the application media stream 150 may be communicated to the participant clients in coordination with the participant media communication streams 180. For example, the application view may be streamed as another “participant” using a peer-to-peer webRTC or some centralized media routing system (e.g., a selective forwarding unit). In some of these variations, the application media stream 150 may be treated specially within a user interface (UI) of an activity session application 160 or as just another participant media stream.

The activity session application 160 functions as a main application used to facilitate access to various activities during a shared collaborative session. The activity session application 160 may further function as the home application that is loaded at the beginning of each new session.

The activity session application is preferably operated at each of a set of participant clients (e.g., computing devices used by participants of an activity session).

The activity session application 160 in one variation can be a web application accessed through a browser. Accordingly, the activity session application 160, in one variation, may be served and operated as a web application on a participant client. In this way, the activity session application 160 may be accessed through a standard browser. Participants may need to login to an account to access the activity session application 160 and join an activity session. Alternatively access a shared link used for an activity session.

The activity session application 160 may alternatively be a native application run on a participant's computing device—a participant client 170 (e.g., personal computer, mobile device, wearable computing device, etc.). In some variations, the activity session application 160 may alternatively be integrated or operated in an alternative manner such as within the shared browser instance 130.

An activity session application 160 may be built to provide different forms of functionality. In one exemplary implementation, the activity session application 160 presents navigable elements (e.g., action inputs) to access different activities. Accordingly, the activity session application 160 may include action inputs that are configured to trigger activation of digital activities, wherein activation of at least one digital activity launches the digital activity on the shared browser instance 130. The media of the digital activity running on the shared browser instance 130 can be streamed and presented in the activity session application 160 of each participant client through the application media stream 150. An action input to launch an activity may be configured to load a specified URL associated with the activity. This URL can be loaded in the browser instance 130.

The activity session application 160 may additionally facilitate other web application functionality supporting access and use of the system by participants. For example, the activity session application 160 (or some connected application) may facilitate participant authentication, administration configuration, and changing sharing/collaboration settings through administration/clinician controls. As shown in FIGS. 10A and 10B, one exemplary implementation may have the activity session application 160 including a main view, sharing/collaboration controls, and administration configuration. The main view may be the main interaction portion and can be used to show a home activity environment or room. Activities may be represented by pictographic icons, activity-description cards, or any suitable user interface element. When an activity is selected, the activity may be presented within this main view. Other approaches to implementing the user interface may alternatively be used.

The activity session application 160 may have different environments or rooms or settings that can be set up for use as the home activity environment for different uses. These differently pre-configured environments may serve as templates. A provider may initially customize different environments for different types of subjects or even individual subjects. The provider may have a default environment that can be used if a customized environment is not available.

An environment may be presented in a variety of ways. In one variation, the environment is presented as a 2D space as shown in FIG. 10A and FIG. 10B, but the environment could be presented as a 3D space or some other visual representation. The activities may be represented by graphical elements (which may be easier for kids to recognize than text menus). The activities, their representation, and/or their placement within the environment may be customized and/or standardized depending on implementation. This may provide a child with a greater sense of familiarity.

As shown in FIG. 11, in another exemplary implementation, the application session application 160 may include a video-chat mode where a core piece of the user interface is used for participants video or audio conferencing. A menu of possible activities may be presented within the user interface as well. Clicking one of the activities may launch a new view that prominently shows a browser instance loaded with a URL of the selected activity. In some cases, native applications may also be possible activities, which may not use the browser instance and instead may be collaborative digital activity using alternative technology for collaboration. The user interface may also provide sharing and collaboration controls. In some variations or instances, activation or collaboration controls may be restricted to a facilitator.

The activity session application 160 may also include other features to facilitate other features. For example, a provider participant may be provided administrative tools to customize and manage multiple environments. For example, a care provider may set up a group of environments to all share common activities, but their placement and arrangement within the rooms could be individually customized for each subject. In this way, environments could be arranged and organized individually, but a care provider will have centralized access to setting activities across multiple subjects.

The activities may include activity links that launch a digital activity within the virtualized computing environment 120. The activities may also include other activity links that trigger other activities which may not depend on or use the virtualized computing environment. For example, native applications (e.g., built in whiteboarding tools, remote monitoring tools synchronized to out of session patient applications, bilateral stimulation tools, other clinical tools, games, etc.) or file-based activities (e.g., opening a media file) may be shown and/or run directly. In one variation, there may be various file-based activities which may display an image, document, video, or audio. As an example, there could be a homework related activity item that can be used to present and review documents completed by or related to the subject participant. In some variations, these homework activities may pull in data from a remote monitoring data system. An application used in viewing the file may have built-in collaboration features implemented outside of the browser instance. However, a browser or application instance may, in some implementations, be used as a way of viewing files. In some variations, the different types of activities may not be noticeable by the participants, as they all could be rendered in a similar manner.

Selection of one of these activities may launch the activity (e.g., a web application) in a full screen or full-view mode. In a full-view mode, the activity may be displayed within the main view of the activity session application 160. In the case where the activity is a natively provided application (e.g., like a whiteboarding application), sharing and collaborative features may be built in natively and the activity may not require launching an activity through the shared browser instance 130. If the activity is a 3rd party web application or a native web application that could benefit from being made collaborative, then the activity may be opened and run in the browser instance 130, and then the media of that activity streamed and presented within a view of the activity session application 160. In this way, an activity from a 3rd party may be presented as being integrated within the activity session application 160 and collaborative features may be enabled through the collaborative sharing features of the system. In some variations, the activity session application 160 may embed each activity such as by using an inline frame (i.e., an “iframe”) or some other approach to embedding web content. State of an activity may be maintained during a session. In some variations, an activity may be refreshed when the activity is left, after a “session” ends, and/or when manually refreshed via user action. The system may include a refresh button (as part of the browser instance 130) to refresh the state of activity. Alternatively state of an activity may be maintained or saved so that it may be accessed at a different time with preserved state.

The participant clients 170 function as the application and/or computing devices used to access and interface with the remote therapy platform. The participant clients 170 in one variation can be a web application accessible through a standard browser. For example, a participant can visit a URL to join a session. A special unique URL may be used to access a particular session. Alternatively, connection to a session may be facilitated after a participant provides clarifying information such as a session ID, username and password, and/or other information. In some characterizations of the system, the participant client 170 may be the same as an activity session application 160.

The system is preferably used when there are participants with asymmetric roles such as a therapist and a subject. In this variation, the set of participant clients comprises at least one participant client associated with a provider participant and at least one participant client associated with a subject participant.

A provider participant client (i.e., a participant client associated with a provider participant) will generally be someone serving the role of facilitator, administrator, therapist, doctor, coach, teacher, or some other role that has more ownership in the experience. In a more simplified version, the provider participant could be the “host” or the account that created or has otherwise been designated as the provider participant client with more authority/privileges.

A subject participant client (i.e., a participant client associated with a subject participant) will generally be someone serving the role of patient, student, trainee, observer, or some other participant in facilitated activity.

The system may also be used where there is no distinction in participant roles.

The system may commonly be used in 1-on-1 sessions but could also be used in group sessions. In a group session, the set of participant clients may include three or more participant clients (e.g., at least three participant clients). In some variations, observers or non-participants may be able to also view or observe a session.

There may be different provider and subject participant client functionality. In particular, the provider participant client (i.e., a provider client) may include functionality specific to this type of participant. In such a variation, a provider participant client may include more administrative and control functionality. For example, the activity session application of the provider participant client may have administrator privileges to at least partially control input processed by the remote shared browser instance. A user interface may be exposed within the provider client or the activity session application 160 for the provider to set the input control permissions of the subject.

In some variations, the system can include a set of participant media communication streams communicated to the activity session application 160 at each participant client 170. The participant media communication streams 180 function as communication media data connection to facilitate communication between the participants. There will generally be a plurality of participant media communication streams 180 enabling real-time video and audio exchange among the participant client devices 170, thereby supporting face-to-face interaction during a session. This could be used to supplement the sharing of a digital activity run on the browser instance 130. In this way, the participant media communication stream 180 may be used so that participants can see and hear each other in addition to seeing and/or hearing content from the shared activity.

Media communication may be established and operated through the system. However, in some variations, the participants may use an outside form of communication while using the digital activity platform. Accordingly, the system may not include and/or interact with the media communications between the participants.

The media communication streams 180 may include video and/or audio streams. As part of or in addition to the participant media communication streams, other data may be streamed or communicated in real-time between participants. For example, application state data of the activity session application 160, participant input data, or any suitable data may be streamed between participants. This may include bidirectional streaming of data or one-directional.

Different data communication approaches may be used in establishing data streams between participants. The media communication streams may use peer-to-peer (P2P) communication, media servers to bridge media streams (e.g., routed through managed or third-party servers), and/or any other suitable type of approach to facilitate real-time media communications between two or more participants. In one exemplary implementation the participant media communication stream may be sent to an intermediary media server such as a selective forwarding unit (SFU) that is configured to forward media to the participant clients 170. In one alternative variation, the system could integrate with 3rd party media streaming service to leverage outside service for managing media streaming.

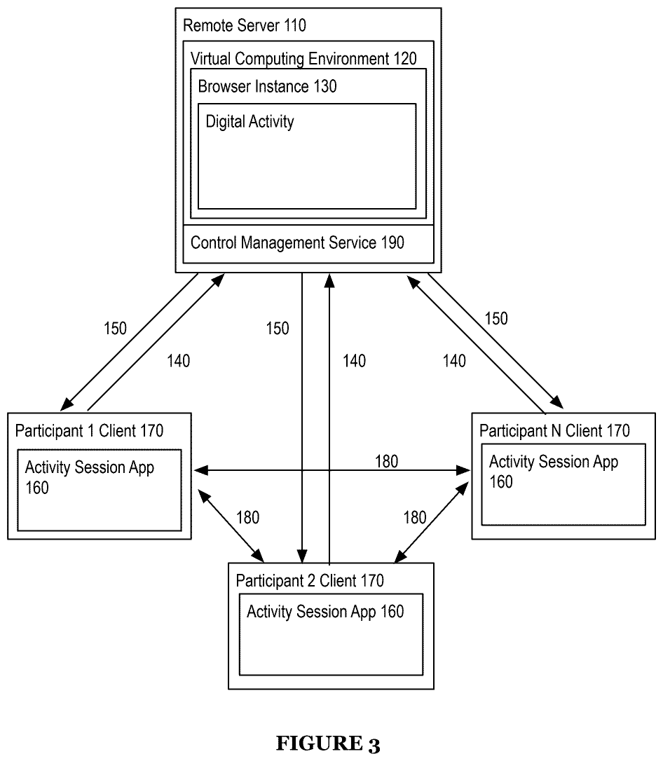

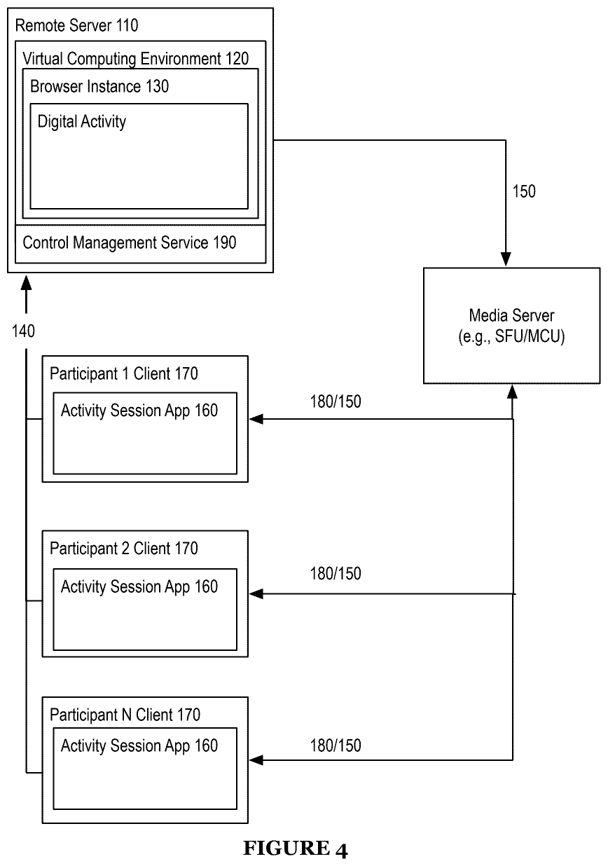

As mentioned, in some variations, the application media stream may be another media stream that is communicated in parallel with the participant media/data. Accordingly, in one variation, the participant media communication streams 180 and the application media stream 150 are communicated through peer-to-peer connections as shown in FIG. 3. In another variation, the system can include or interface with a media server configured to receive the participant media communication streams and the application media stream, and to forward received media streams to the participant clients as shown in FIG. 4. The media server may act as an intermediary that forwards participant media and/or application media to participants. The media server may optimize streaming for connectivity and performance. For example, the media server may be a selective forwarding unit.

Various communication topologies may be used to manage media communication between the participants and the application media. In one variation, the video and audio from participants may be communicated to the remote server. In an MCU (Multipoint Control Unit) architecture, the communication media may be mixed with the application media content so that a single media stream can be sent to other participants.

A control management service 190 functions to manage coordination of input from multiple participants. The control management service 190 can be operable in connection with the remote shared browser instance 130. The browser instance 130 will generally be configured to respond to a single form of mouse and keyboard input. The control management service 190 can convert multiple sources of input into a singular input for the browser instance 130 that can be used to control the current activity. The input interaction channels 140 communicated from each participant clients 170 are routed to the control management service 190 which is then configured to use the received inputs in controlling input to the browser instance 130. In particular, the control management service 190 is configured to selectively use mouse input from input interaction channels of the set of participant clients. Various rules or logic may be used to determine how to direct input to the browser instance 130 from two or more sources of input.

As shown in FIG. 2, the control management service 190 may, in one variation, be configured to update input control based on the states of input interactions. When one participant sends some input interaction, the control management service 190 can check the state of interaction locks to see if input interactions should be locked to a participant, if the input interactions should be ignored because of an existing interaction, or if an interaction lock should be ended. If the interaction is an “in progress” engagement, then the input control may be temporarily locked to the participant. If another participant has acquired an interaction lock, then the inputs from other participants can be ignored until completion of the interaction lock. If an interaction represents a “completed” engagement for the particular interaction lock, such as a mouse up event, then the interaction lock can be released or otherwise ended so that other participants may direct the input. The control management service 190, in some variations may be configured to implement such logical checks. As shown in FIG. 4, the control management service 190 may be enacted to determine how to deal with different interactions from different participants depending on the state of interaction. This logic may work with two or more participants.

In some cases, cursor control in particular may be regulated through the control management service 190. Cursor control can be used to control positioning or movement of a cursor and mouse events (right and/or left mouse click down and up, scrolling, and/or any suitable cursor input). This may also support dealing with other comparable inputs such as touch inputs from a touch-based input device.

The control management service 190 may additionally selectively enforce different permissions. The control management service 190 may allow one or more participants to manage control permissions of one or more other participants. For example, a therapist could turn on and off the option for a child to control input. This may be used so that the therapist can take control over the interactions at any time. Other forms of input permissions may be used. For example, a therapist could define certain regions or activity elements (e.g., buttons or web elements defined in the DOM) where a child may or may not provide input.

These components may be used in facilitating single activity sessions. However, the system can generally support multiple distinct activity sessions, each with their own set of participants. As discussed, this may include different combinations of types of participants. For example, there may be three participants in one activity session with one provider participant and two subject participants as well as a second activity session for two provider participants and one subject participant. In general, the system may scale to support an arbitrary number of activity sessions, each with an arbitrary number of provider and subject participants.

In terms of a provider participant controlling input control, the provider participant may be provided with functionality to select which participant has the ability to interact with a browser instance at any one time. In another variation, all participants may have control dynamically controlled where a user with active control has locked control until its released (e.g., by becoming idle). As also discussed herein, multiple cursors may be supported.

As shown in FIG. 5, the system may include an arbitrary number of servers to serve an arbitrary number of activity sessions.

As also shown in FIG. 5, the system may also have a server instantiate multiple isolated virtual computing environments 120 for multiple browser instances 130.

Flexibility in allocating servers, isolated containers, and browser instance(s) 130 may be used to support various functionality. For example, for group activity sessions with three or more participants, it may be useful to have the group to break out into individual activities or sub-group activities. These breakout sessions may be achieved by initiating different browser instances (in different or the same virtual computing environment 120) where each participant can interact with their respective activity during the breakout session. A provider participant may move between these different breakout sessions—changing the browser instance the provider participant interacts with. As shown in FIG. 9, the participants may move between media connections of the group browser instance and breakout session browser instance.

In some variations, the system may also include an interactivity layer. An interactivity layer can be a user interface overlaid within the activity session application. In particular, the user interface of the interactivity layer may be a layer overlaid on the rendering of the browser instance 130 (i.e., the media of the shared browser instance streamed and presented in the activity session). The interactivity layer can be a UI layer rendered in the activity session application 160. When used for real-time interactions, any suitable real-time collaboration data protocol may be used to synchronize state of the interactivity layer. The interactivity layer may provide additional collaborative functionality. It may be configured for real-time interactions that are separate from interactions with the browser instance 130. For example, the interactivity layer may be used to display where a participant cursor is located when the participant does not have active control of the browser instance. This may be used so that a participant can “point” or use a cursor to gesture. For example, a child may be able to point at a section of the user interface in the browser instance. The interactivity layer may also be used for other collaborative features like displaying reactions or emotes. For example, a therapist may add a thumbs up emoji to signal a good job to a child. As yet another example, the interactivity layer may be used as a “whiteboard” layer where annotations (drawing, shapes, graphics, text, etc.) can be added over the rendering of the browser instance.

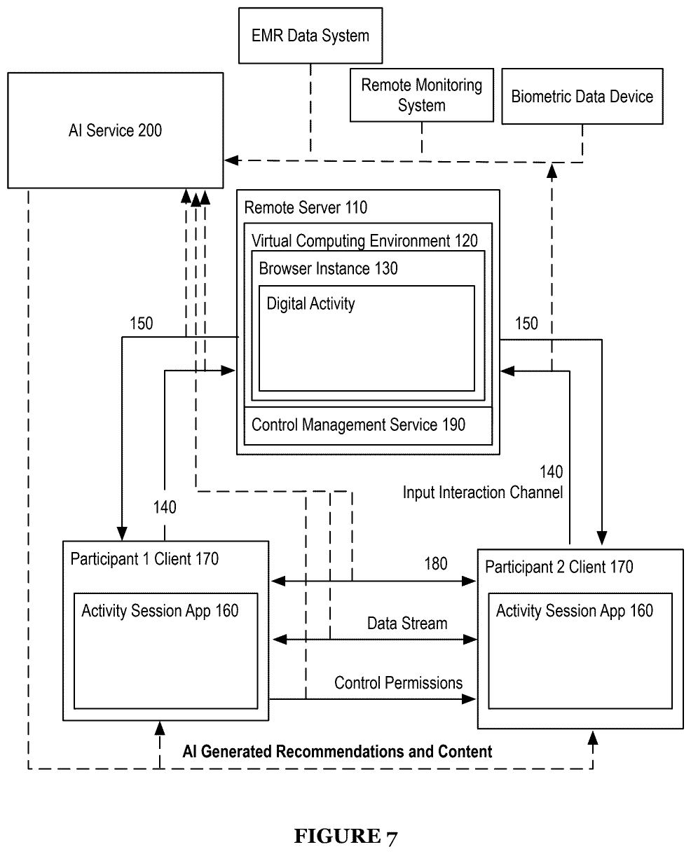

In some variations, the system may include, interface, or otherwise integrate with an AI service 200 (i.e., AI engine). Various data channels of the system can interface with the AI service to provide automated AI-enhanced functionality. In some variations, the data from the input interaction channel, the application media stream, and/or participant media communication stream is supplied as input to the artificial intelligence service with configuration to generate an output as shown in FIG. 7. The output may be a recommendation, an analysis assessment, a generated asset, and/or achieve some other task.

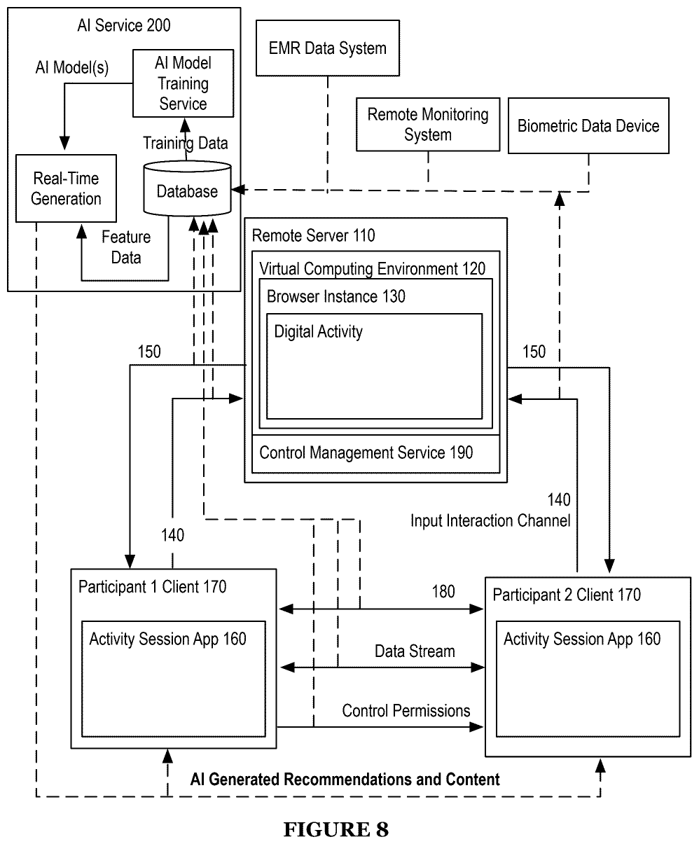

In one variation, the AI service 200 may be directly integrated into the system as shown in FIG. 8. The AI service 200 can include a database that stores collected feature data, an AI training service and/or an AI processing engine. Collected data stored in the database may be fed into various AI processing engines for analysis and result output. The collected media may include communication media from the participants, input interactions from the participants, analysis and data from activity applications, and/or other sources of data from use of the digital activity platform like electronic health or health related data, or biometric data from personal health devices. The collected data may additionally or alternatively be supplied as training data for an AI model training service. AI models updated or resulting from the training can then be used by the AI processing engines. AI may be used in different ways. As such there may be different AI processing engines with different AI models. AI models may be trained and/or the AI service may be used for analysis, training, and/or generation.

In other variations, an external AI service 200 may be used. The system may interface with an external large language model or other type of AI service 200 using an application programming interface (API) or any suitable programmable interface. Data can be transmitted to the external AI service 200, which can then return some result.

One or more AI services 200 may also be used for performing or achieving different tasks. Any suitable AI data pipeline may be used to perform arbitrarily complex analysis and synthesis from collected data.

In some exemplary variations, the AI service 200 may be used for auto-generated treatment planning, session preparation, (e.g., activity/intervention selection), clinical documentation, and for other automated generation tasks. Automating such tasks may be beneficial to address challenges like clinical burnout due to high administrative burden and the public health need for higher clinical efficiency amidst a severe shortage of mental health professionals. In another variation, an AI service 200 may be used to generate content for customized experiences for a participant.

In some variations, the system may also interface with various data sources. The data sources may be used as a source for data that can be used to augment operation of the system. The data sources may alternatively be updated with data generated from the system.

In one such variation, the system includes an interface to an EHR data system. In a system variation that includes an integration to an AI service 300, data from the EHR data system may be supplied as at least one input to the AI service 200. In one example, electronic health record data may be used to augment generated recommendations that can be used to transform the activity session of a subject participant in some way.

The EHR data system may also be updated based on data from the system. For example, the AI service 200 may generate an assessment of a subject participant. This assessment may facilitate the completion of some standardized questionnaires or tests, which may be automatically updated in the EHR data system for the subject participant.

In another variation, the system includes an interface to biometric device data. The biometric device data can be associated with at least one participant of the participant clients. The biometric device data may be data from a health device worn or used by a participant. A biometric device data could come from a heart monitor, a respiratory monitoring device, or any suitable type of device. In some variations, the biometric device data is further supplied as input to the artificial intelligent service. Other alternative data inputs may also be used in addition or as an alternative to the biometric device data and EHR data systems.

Similarly, the system may include an interface to any suitable alternative data source. In one variation, the system may additionally include an interface to a remote monitoring system. The remote monitoring system can be associated with at least one participant of the participant clients. A remote monitoring system could be an external tool or system used to provide supplemental external data about a subject. In one example, the remote monitoring system could be an application (e.g., native/integrated app, 3rd party app, or otherwise) where patients can perform common homework protocols, such as thought records, food diary, exposure logs, dairy cards, journals, and the like. As with other supplemental data sources, data of the remote monitoring system may be supplied as input to the artificial intelligence service.

### 3. Method

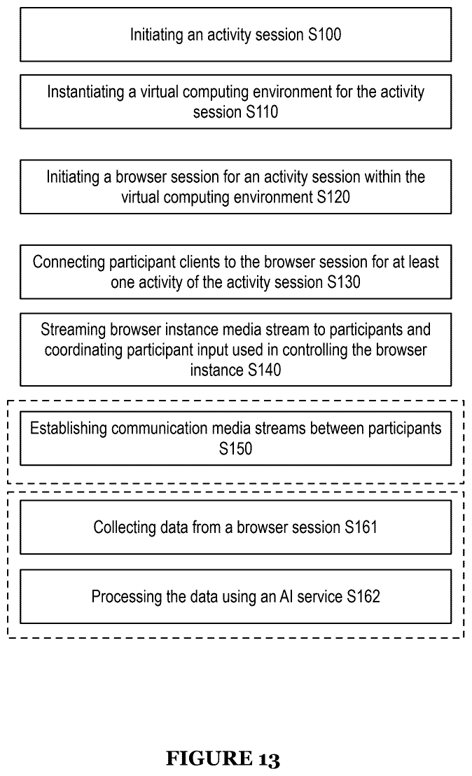

As shown in FIG. 13, a method for operating a digital activity platform can include initiating an activity session S100; instantiating a virtual computing environment for the activity session S110; initiating a browser for an activity session within the virtual computing environment S120; connecting participant clients to media from the browser instance the browser session for at least one digital activity of the activity session S130; and streaming and coordinating participant input used in controlling the browser instance S140. In some variations, the method may additionally include establishing communication media streams between participants S150. In some variations, the method may include collecting data from a browser session S161 and processing the data using an AI model S162.

These method variations may be implemented for supporting a single activity session but may additionally be used through a method supporting multiple instances. Such method variation can include a cloud platform hosting a set of servers that support multiple activity sessions that each support remote collaboration of multiple participants within a digital activity, wherein each activity session comprises a process of initiating an activity session S100; instantiating a virtual computing environment for the activity session S110 on one of the set of servers; initiating a browser for an activity session within the virtual computing environment S120; connecting participant clients to media from the browser instance the browser session for at least one digital activity of the activity session S130; and streaming and coordinating participant input used in controlling the browser instance S140.

The method is preferably implemented by a system substantially similar to the one described herein, but any suitable alternative system may alternatively be used.

Block S100, which includes initiating an activity session, functions to start an activity session for collaborative communication of multiple participants. The activity session, in one variation, can be initialized within an activity session application or some other suitable participant client as described herein. In one variation, participants may access the activity session application as a web application within a browser. In another variation, participants may use a native application that is or includes the activity session application. Initiating an activity session will preferably start with at least one participant (e.g., a provider participant). Additional participants (e.g., subject participant or another provider participant) can join the activity session.

An activity session application preferably includes a home or starting state that presents some environment or user interface with a set of different activities. The activities may be presented as selectable graphical elements within the environment. Alternatively, the activities may be presented as a menu of different digital activities. The activities may be configured with a URL of a digital activity that can be loaded in a browser instance. Selection of an activity loads the activity full screen within the main view. In an alternative implementation, an activity session may be initiated with settings for immediately loading a particular digital activity in a browser instance.

Initiating the activity session may also include initiating with selected session configuration. The session configuration may define different options such as the presentation of the environment, types of activities available, and/or arrangement/presentation of the activities. When starting a new session, a provider participant may be able to indicate the session configuration to be used for the session. In one example, different children may each have an assigned “environment”. By clicking/selecting an environment of a child, a therapist could start a new session for that child.

Other features may also be supported such as sharing an activity session through a link, scheduling an activity session, inviting participants to an activity session, or other suitable features.

Block S110, which includes instantiating a virtual computing environment for the activity session, functions to create a virtualized computing environment for running one or more shared remote activities for an activity session. As discussed, the virtualized computing environment in one variation can be a container such as one created using Docker or other containerization technology. In another variation, the virtualized computing environment could be a virtual machine. The virtualized computing environment can be initiated and established on a remote server. As an alternative approach, in some variations, the method may not use virtualized computing environments and initialize browser sessions directly within the native computing environment of a server.

In one variation, a virtualized computing environment is preferably used for each activity session. Accordingly, initiating a virtualized computing environment may be performed for each new activity session. In some variations, a new virtualized computing environment may be established and initialized in response to a new session event. In some alternative variations, a pool of initialized virtualized computing environments may be established and waiting for use and/or assignment by a session. For example, a pool of virtualized computing environments may be set up and established within a server or a set of servers. When a provider selects an option to start a new session, then one of those virtualized computing environments may be assigned to the session.

Virtualized computing environments may be used because they create separation between the data of all other participants. The interactions within the virtualized computing environment may be more private and secure. Additionally, after the session, the virtualized computing environment may be deleted or otherwise removed or reset, which can further protect the PII and PHI data of a participant.

Block S120, which includes initiating a browser session for an activity session within the virtual computing environment, functions to initiate the browser session for running one or more different digital activities and adding collaborative features to the different activities during an activity session. The browser session is preferably initiated and run within a virtualized computing environment established through block S110. If no virtualization is used, then the browser session may alternatively run within the computing environment of a server.

The digital activities will generally be accessed through a URL or link that can be opened by the browser instance. As such initiating the browser session for an activity session may include opening the browser to a web application URL of a digital activity launched as part of the activity session. In many variations, a digital activity will be launched in response to a selection of some activity from within an activity session application. Accordingly, the method may also include, from an activity session application running on a participant client connected to the activity session, receiving an activity activation event associated with some link that thereby triggering opening the browser to the link.

Block S130, which includes connecting participants simultaneously to the browser session for at least one activity of the activity session, functions to connect multiple participants to a shared presentation of an activity during the activity session. All participants will preferably connect to the browser session and gain collaborative access to the activity. Connecting participants may include establishing, for each connecting participant, an input interaction channel and an application media stream. An input interaction channel can be established between each activity session application of a participant client to the remote browser. This may be used to communicate input control data from a participant to the browser. The application media stream is preferably a media stream of the interface of the browser instance that is streamed back to the participants.

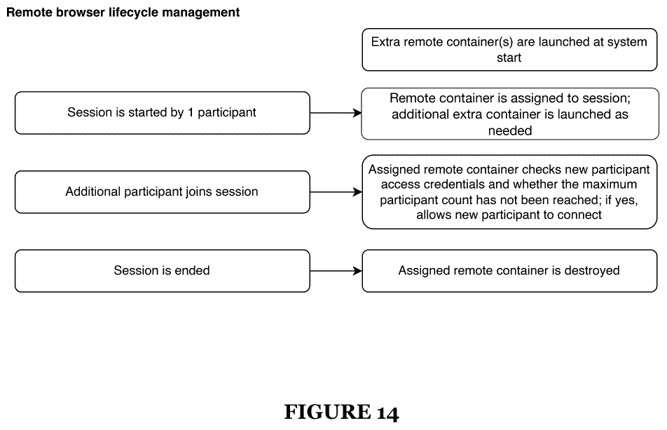

In some variations, connecting participants can happen independently of each other. Additionally, participants may be authenticated before providing access to a session. As shown in an exemplary life cycle diagram in FIG. 14, one participant (e.g., a therapist) may start a session. This initiating action can assign a session to a virtualized computing environment. A new unassigned virtualized computing environment may be launched to replace the newly assigned virtualized computing environment in the pool of unassigned virtualized computing environments. Then one or more additional participants may make requests to join the session, and they can be added. For all participants, authentication may be performed to verify they are authorized to connect to that particular session. When a session is ended, the virtualized computing environment along with the associated local data may be destroyed. As mentioned, in some variations, a session may be kept active for longer than one working session between participants. For example, if a therapist wants to restart an activity in its current state with a child during their next appointment, then the session may be maintained so that the therapist and child can start right where they left off.

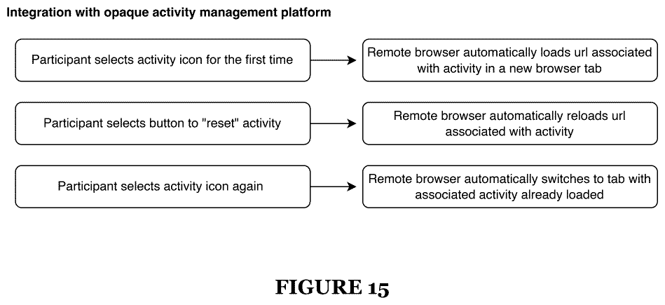

In connection with block S130 and S140, the method can include managing application state within the browser instance. Managing application state can involve changing the state and presentation of different activities. The activities preferably represent different web applications. Accordingly, they may represent a particular URL or address used to access the web application. In one exemplary variation shown in FIG. 15, when a participant selects an activity for the first time from an activity session application, the browser can be instructed to load the URL associated with the digital activity. The URL is preferably loaded within a separate view which can be presented full-screen or “full-view” (e.g., presented within some main sub-region of the activity session application) within the user interface. To the participants, it may appear that a new activity was launched. In some variations, there can be a reset or refresh button. When a participant presses the reset button the browser instance can reload the URL of the current activity. There may be a navigational element that allows a user to navigate back to the home environment. Navigation to the home environment preferably does not change the state of the activity within its view, but it may end the presentation of the browser instance to the participants. In other words, the state of the launched web application can be maintained. If a participant selects an activity again, the browser instance can switch to presenting the view of the activity.

Block S140, which includes streaming and coordinating participant input used in controlling the browser instance, functions to coordinate multiple inputs from different participants in simulating a singular input source for interacting with the activities. Controlling participant input for a shared activity will generally include monitoring multiple inputs from different participants and updating a final input control for the browser instance based on states of input interactions of the multiple inputs. The final input control is preferably the simulated input supplied to the virtualized computing environment and the browser instance. In some variations, the states of input interactions used for updating the final input control may depend on the type of inputs received, the timing of the inputs, the activity elements receiving the interactions, and/or other factors.

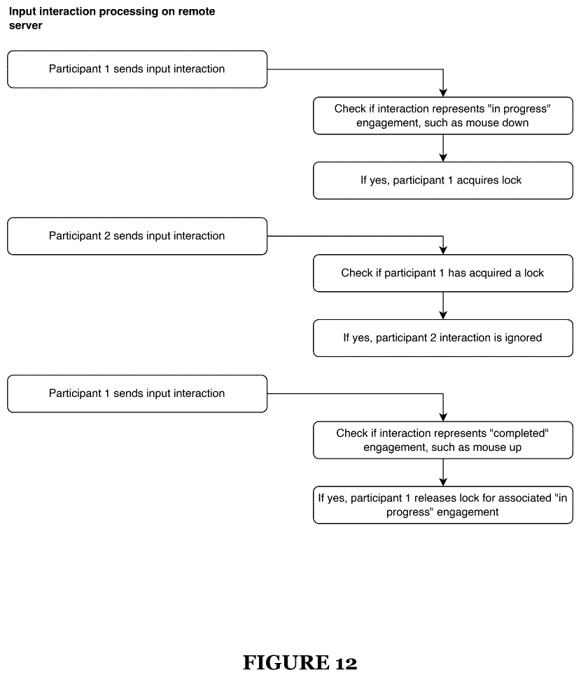

In one exemplary variation, as shown in FIG. 12, controlling participant input for a shared activity may include monitoring multiple inputs from different participants and updating a final input control for the browser instance based on engagement lock status. An input interaction may include a change in mouse position, activation or deactivation of some mouse button/action (e.g., right click down, right click up, left click down, left click up, scroll wheel up/down, etc.), touch events, and/or some keyboard action.

In response to each input interaction monitoring the multiple inputs from different participants can include verifying engagement lock status which can include checking if engagement is locked (e.g., indicating another participant has temporary control of input during some interaction). If there is no current engagement lock by another participant, then if the interaction of the input interaction is an “in progress engagement” then the engagement may be locked and that participant gains control of input during their interaction engagement. If an interaction represents a completed engagement (e.g., a complimentary action to an in-progress engagement like a mouse up event), then the engagement lock can be released/ended. This functions to lock interaction and trade control when a user takes some explicit action. If a user makes a mouse down action when there is no engagement lock, then the location of their mouse cursor and the clicking action can be transferred to the activity thereby resulting in a mouse down action at that location. The engagement lock can be sustained until some complimentary action is detected like a mouse up event. In this way, if a user clicks an object to drag it, then the participant retains input control until they release the mouse button, ending their interaction. When a participant is controlling input, additional locks may also be added for that participant for different actions, and the other participant can only interact after all locks are released.

In some variations, the server, virtualized computing environment, and/or browser may support multiple cursors in which case the human interface device input of a participant may be mapped to one of a set of multiple cursors.

In some variations, keyboard inputs may be streamed as they are received without any controlled management.

Controlling participant input for a shared activity may additionally include enforcing control permissions according to administrator controls. In some variations, the provider participant may be provided some administrative tools to disable, limit, and/or otherwise adjust input capabilities of one or more participants. For example, a therapist may temporarily disable input of a child until it's the child's turn to interact with an application.

For example, certain regions, buttons, or elements of an application may have different input permissions. These may be set per activity or across a session. For example, only a child participant may be blocked from using their inputs to select certain options in an activity, but they could use their mouse within a main “game region” (e.g., possibly using DOM based rules).

Block S150, which includes establishing communication media streams between participants, functions to establish video and/or audio communication between the participants. Additionally data streams used for coordinating application state or other functionality may also be streamed. This may include negotiating and establishing streaming media connections between the participants. The media can be presented as a video call. In some variations, this may be directly integrated into the participant client interfaces. In some variations, peer-to-peer (P2P) media streaming can be established. In such cases, the virtual computing environment may serve as one of the peers where the application media stream (e.g., audio/visual content of the browser instance) may be part of a P2P media mesh network. In some variations, the media may be bridged by a remote server (e.g., a media server like an SFU), possibly the same remote server used for the virtualized computing environment. In some variations, media of the application media stream and media received from a participant may be mixed and combined such that a single output media stream may be used for each participant (e.g., a media server like an MCU). Various communication approaches may be used to establish the communication media streams.

Blocks S161 and S162, which include collecting data from a browser session S161 and processing the data using an AI service S162, functions to integrate AI processes into operation of the digital activity platform. As an initial step data can be collected from a session. The data may include data related to or including communication media, input interactions, and/or application/activity state. The data collected may reflect the nature of the participant's verbal and visual interactions. The data collected may also reflect the types of activity from the activity side as well as how a participant is interacting with the activity.

The method may also include interfacing with other data sources such as EHR data systems or biometric data devices. Data from such sources may also be used as inputs to an AI service.

In some variations, the data collected can be used as an input supplied to an AI service. In the case of an LLM AI service, the data input may be included in context data along with a prompt for generating some result. In some cases, an external AI service may be used. In other variations, a hosted AI model may be used. Variations employing AI modeling may additionally include training or updating an AI model from the collected data.

Processing data using an AI service may be used for various activities such as generating session activity documentation. Session activity documentation may leverage AI service for automatic tracking of activity usage and performance in activities. Large language models, visual models, and/or multi-modal models may be used to analyze and summarize content of activities. Additionally, plans for treatments and/or activities may be generated based on the analysis. In some examples, processing data using an AI service may be leveraged within the interactions and interface of the activity session platform. For example, activity recommendations, customized activities, and/or customized interfaces may be generated. Additionally or alternatively, processing data using an AI service may be used outside of the activity session interface. For example, AI generation, summarization, and/or analysis may be used to improve workflows (e.g., providing clinical documentation), enable measurement-based care (e.g., treatment engagement measurements), provide summaries or other updates about care to participants and other interested parties, and/or generate homework or other tasks and check-ins outside activity sessions. Other applications for generation and/or analysis may similarly be used.

For collective data analysis and usage across a pool of different participants, the method may include anonymizing the data such that no personally identifiable information is used.

### 4. System Architecture

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In one variation, a system comprising of one or more computer-readable mediums (e.g., non-transitory computer-readable mediums) storing instructions that, when executed by the one or more computer processors, cause a computing platform to perform operations comprising those of the system or method described herein such as: initiating a virtualized computing environment for a participant session, initiating a browser session to an activity session application, connecting participant clients to the browser session, and controlling participant input for a shared activity.

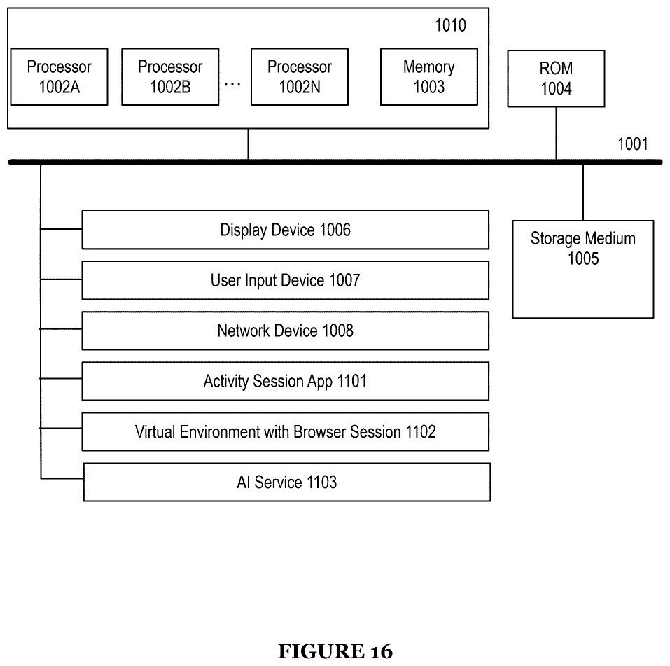

FIG. 16 is an exemplary computer architecture diagram of one implementation of the system. In some implementations, the system is implemented in a plurality of devices in communication over a communication channel and/or network. In some implementations, the elements of the system are implemented in separate computing devices. In some implementations, two or more of the system elements are implemented in the same devices. The system and portions of the system may be integrated into a computing device or system that can serve as or within the system.

The communication channel 1001 interfaces with the processors 1002A-1002N, the memory (e.g., a random-access memory (RAM)) 1003, a read only memory (ROM) 1004, a processor-readable storage medium 1005, a display device 1006, a user input device 1007, and a network device 1008. As shown, the computer infrastructure may be used in connecting an activity session application 1101, virtualized computing environment with browser instance 1102 as described herein, an AI service 1103, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid-state drive, a ROM, an EEPROM, an electronic circuit, a semiconductor memory device, and the like. The processor-readable storage medium 1005 can include an operating system, software programs, device drivers, and/or other suitable sub-systems or software.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeably without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A system comprising:
a remote server;
at least one virtualized computing environment instantiated on the remote server;
at least one remote shared browser instance executed within the at least one virtualized computing environment; and
a set of participant clients connected to the remote shared browser instance through an input interaction channel that communicates inputs from the set of participant clients to the remote shared browser instance and an application media stream which communicates media from the remote shared browser instance to the set of participant clients.

2. The system of claim 1, further comprising an activity session application that is operated at each of the set of participant clients.

3. The system of claim 2, wherein the activity session application comprises action inputs that are configured to trigger activation of activities, wherein activation of at least one activity launches the activity on the shared browser instance and the media of the activity running on the shared browser instance is streamed and presented in the activity session application of each participant client through the application media stream.

4. The system of claim 2, wherein the set of participant clients comprises at least one participant client associated with a provider participant and at least one participant client associated with a subject participant.

5. The system of claim 4, wherein an activity session application of the provider participant has administrator privileges to at least partially control input processed by the remote shared browser instance.

6. The system of claim 4, wherein the set of participant clients comprise at least three participant clients.

7. The system of claim 1, further comprising a set of participant media communication streams communicated to the activity session application at each participant client.

8. The system of claim 7, wherein the participant media communication streams and the application media stream are communicated through peer-to-peer connections.

9. The system of claim 7, further comprising a media server configured to receive the participant media communication streams and the application media stream, and to forward received media streams to the participant clients.

10. The system of claim 1, further comprising a control management service operable in connection with the remote shared browser instance and configured to coordinate input commands from the input interaction channels of the set of participant clients for input used at the remote shared browser instance.

11. The system of claim 10, wherein the control management service is configured to selectively use human interface device input from input interaction channels of the set of participant clients.

12. The system of claim 1, wherein the isolated environment is a container environment.

13. The system of claim 1, wherein the isolated environment is a virtual machine environment.

14. The system of claim 1, further comprising an interactivity layer visually overlaid within the activity session application.

15. The system of claim 1, further comprising an interface to an artificial intelligence service.

16. The system of claim 1, wherein the data from the input interaction channel and the application media stream is supplied as input to the artificial intelligence service with configuration to generate a recommendation.

17. The system of claim 16, further comprising an interface to an electronic health record data system, and wherein data from the electronic health record data system is further supplied as input to the artificial intelligence service.

18. The system of claim 16, further comprising an interface to biometric device data associated with at least one participant of the participant clients, and wherein the biometric device data is further supplied as input to the artificial intelligence service.

19. The system of claim 16, further comprising an interface to a remote monitoring system associated with at least one participant of the participant clients, and wherein data of the remote monitoring system is further supplied as input to the artificial intelligence service.
